(19) United States
(12) Patent Application Publication
(10) Pub. No.: US 2022/0348526 A1
(43) Pub. Date: Nov. 3, 2022

(54) Improved process for preparing cyclopropyl compounds from alkenes

(71) Applicant: SALTIGO GmbH, Leverkusen (DE)

(72) Inventors: Andre Grossmann, Cologne (DE); Bjorn Schlummer, Bonn (DE)

(73) Assignee: SALTIGO GmbH, Leverkusen (DE)

(21) Appl. No.: 17/642,444

(22) PCT Filed: Sep. 9, 2020
(86) PCT No.: PCT/EP2020/075207

§ 371 Date: Mar. 11, 2022

(30) Foreign Application Priority Data
Sep. 12, 2019 (EP) 19196901.3

(51) Int. Cl. C07C 29/44

(52) U.S. Cl. CPC C07C 29/44; C07C 2601/02

(57) ABSTRACT

The invention relates to an improved process for preparing cyclopropyl compounds from alkenes through reaction of the alkene in the presence of bromochloromethane, elemental zinc, and elemental copper or copper compounds (cyclopropanation reaction).

The invention relates to an improved process for preparing cyclopropyl compounds from alkenes through reaction of the alkene in the presence of bromochloromethane, elemental zinc, and elemental copper or copper compounds (cyclopropanation reaction).

The conversion of alkenes to the homologous cyclopropyl compounds is known under the name Simmons-Smith reaction, in which normally dihaloalkanes, usually diiodomethane, are reacted in the presence of zinc and copper. In this reaction, a zinc carbene (carbenoid) is generated from the copper-activated zinc (zinc-copper couple) as an intermediate, the methylene group of which undergoes addition to the double bond of the alkene.

Whereas cyclopropanation reactions in the presence of diiodomethane often give high yields as a consequence of the high reactivity of the latter, which is indeed necessary for the formation of the zinc carbene intermediate, reactions of chlorine- or bromine-containing alkanes as carbene precursors are generally much less favorable. Variants of this reaction are accordingly described in the prior art, for example the use of dibromomethane as the dihaloalkane; in these variants, the zinc or the zinc-copper couple is additionally activated by sonication (J. Org. Chem. 50, 1985, 4640; Friedrich et. al.), by titanium tetrachloride catalysis (J. Org. Chem. 1989, 54, 2388; Friedrich et al.) or by sodium dihydridobis(2-methoxyethoxy)aluminate (SDBA) (EP 0321409 A2).

There are also variants for reactions with bromochloromethane as the dihaloalkane that afford the desired cyclopropyl compounds in acceptable yields only with additional activation of the zinc or zinc-copper couple.

In J. Org. Chem., 56 (10), 3255, 1991 (Sibille et al.), an electrochemical cyclopropanation of allyl alcohols with dibromomethane or bromochloromethane using a zinc anode and a carbon fiber cathode is for example described. However, this reaction has the disadvantage of being costly, laborious and difficult to control when carried out on a relatively large industrial scale.

As prior art, WO 2017/024126 additionally describes a process in which dibromomethane or bromochloromethane are used as dihaloalkanes instead of diiodomethane and in which the zinc or zinc-copper couple is additionally activated. Haloalkylsilanes, for example chlorotrimethylsilane, are here absolutely essential as activators in order to obtain acceptable yields. The silicon-containing activators that are required mean that this process is likewise more laborious and more costly to carry out on a larger scale than the classic Simmons-Smith reaction, which is itself uneconomical on a relatively large scale because of the high price of the diiodomethane that is required.

The cyclopropyl compounds prepared by the process according to the invention are for example intermediates or end products for various commercial uses, such as flavorings or agrochemicals. The compound of formula (II) is for example an intermediate for the azole fungicide cyproconazole (DE 3406993, CN 105820128, CN 101857576).

There is thus still the need to provide, and hence the technical problem of providing, a variant of the Simmons-Smith reaction that does not have the disadvantages of the prior art.

Surprisingly, a process has now been found for preparing cyclopropyl compounds that comprises the reaction of the corresponding alkene, which contains at least one carbon-carbon double bond, with bromochloromethane in the presence of (i) elemental zinc, (ii) catalytically active amounts of elemental copper and/or copper(I) compounds and/or copper(II) compounds, and (iii) at least one solvent, characterized in that the reaction of the alkene takes place in the absence of organosilicon compounds, preferably in the absence of haloalkylsilanes, and/or without addition of organosilicon compounds, preferably without addition of haloalkylsilanes, and that the elemental zinc has a lead content of not more than 0.005% by weight (50 ppm), preferably of not more than 0.002% by weight (20 ppm).

The alkenes used for the purposes of the invention are organic compounds having at least one carbon-carbon double bond. This carbon-carbon double bond is for the purposes of the invention an aliphatic double bond and not an aromatic double bond. The alkenes may be linear, branched or cyclic. The aliphatic carbon-carbon double bond may be terminal or non-terminal. If the alkene contains more than one carbon-carbon double bond, these may be conjugated or unconjugated. The alkenes used in the process according to the invention may bear further substituents such as further aliphatic or aromatic radicals, which may in turn be likewise substituted or unsubstituted.

The alkene used is preferably the compound of formula (I),

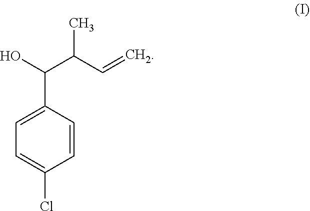

In the cyclopropanation, the alkenes give rise to the formation of the corresponding homologous cyclopropyl compounds, which are to be understood formally as products of the addition of a carbene “CH₂” to the double bond of the alkenes. When the compound of formula (I) is used by preference as the alkene, the compound of formula (II) is formed as the cyclopropyl compound,

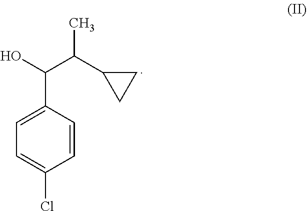

The bromochloromethane (CH₂BrCl) used as the haloalkane is commercially available or can be prepared in-house using suitable processes and preferably has a purity of at least 98% by weight and a water content of less than 0.02% by weight, preferably of less than 0.007% by weight.

The bromochloromethane used as the haloalkane may also be used for example as a mixture with dibromomethane. However, the reaction becomes more uneconomical as the proportion of dibromomethane increases. In addition, the yields achieved decrease as the proportion of dibromomethane increases.

The process according to the invention is carried out in the presence of elemental zinc. The elemental zinc is normally present in finely divided form. Finely divided form is for the purposes of the invention understood to mean a powdery, fine-grained or granular solid that is preferably easily pourable and therefore easily meterable. A prerequisite for the process according to the invention is that the elemental zinc has a lead content of not more than 0.005% by weight, preferably not more than 0.002% by weight. The lead content in this concentration range is normally measured using atomic absorption spectrometry.

The process according to the invention preferably uses elemental zinc in finely divided form, i.e. as elemental zinc having a particle size distribution D90ₘₐₛₛ of not more than 0.5 mm. The particle size distribution D90ₘₐₛₛ of not more than 0.5 mm means for the purposes of the invention that 90% by weight of the sample to be measured has a particle size of 0.5 mm and smaller. The particle size distribution D90ₘₐₛₛ is normally determined by sieving and subsequent weighing of the sieved particles and has high accuracy. In a further embodiment, the metallic zinc preferably has a sieve distribution of not more than 15% by weight having a particle size of greater than 250 μm, of 25% to 50% by weight having a particle size of from 150 to 250 μm, of 30% to 60% by weight having a particle size of from 45 to 150 μm, and not more than 15% by weight having a particle size of less than 45 μm, but wherein a particle size distribution D90ₘₐₛₛ of not more than 0.5 mm is conformed to overall.

Besides a lead content of not more than 0.005% by weight, preferably of not more than 0.002% by weight, the elemental zinc further preferably has a total content of other metals of not more than 1% by weight, preferably 0.1% by weight, more preferably 0.05% by weight. These other metals are for example cadmium, iron, mercury, bismuth or indium. The contents of the other metals are generally determined by atomic absorption spectrometry.

The elemental zinc further preferably has a zinc content of at least 99.0% by weight, preferably of at least 99.9% by weight. The zinc content is generally determined by atomic absorption spectrometry.

In the process according to the invention, from 1.5 to 4 mol, preferably from 2 to 3 mol, of metallic zinc based on 1.0 mol of the alkene, preferably based on 1.0 mol of the compound of formula (I), is preferably used.

The process according to the invention uses either elemental copper, copper(I) compounds or copper(II) compounds, or mixtures thereof.

Besides a lead content of not more than 0.005% by weight, preferably of not more than 0.002% by weight, the elemental copper preferably has a total content of other metals of not more than 1% by weight, preferably of 0.1% by weight, more preferably of 0.05% by weight. These other metals are for example cadmium, iron, mercury, bismuth or indium. The contents of the other metals are generally determined by atomic absorption spectrometry.

When copper(I) compounds are used in the process according to the invention, this is preferably copper(I) chloride. When copper(II) compounds are used in the process according to the invention, these are preferably copper(II) chloride, copper(II) phosphate, copper(II) carbonate or mixtures thereof.

The amount of copper or copper compound used depends on the number of carbon-carbon double bonds in the alkene that are to undergo cyclopropanation. If the alkene has only one double bond that is to undergo cyclopropanation, the catalytically active amount of elemental copper and/or copper(I) compounds and/or copper(II) compounds is used in a total amount of from 0.001 to 0.1 mol, preferably from 0.001 to 0.01 mol, based on 1.0 mol of the alkene, preferably based on 1.0 mol of the compound of formula (I). For every further mole of aliphatic carbon-carbon double bond in the alkene that is to undergo cyclopropanation, the corresponding whole-number multiple of the abovementioned amount is used.

The amount of bromochloromethane used in the process according to the invention also depends on the number of double bonds in the alkene that are to undergo cyclopropanation. Where there is one double bond in the alkene, as is the case for example in the compound of formula (I), from 1 to 3 mol, preferably from 1.7 to 2.2 mol, of bromochloromethane based on 1.0 mol of the alkene, preferably based on 1.0 mol of the compound of formula (I), is used for example. For every further mole of carbon-carbon double bond in the alkene that is to undergo cyclopropanation, the corresponding whole-number multiple of the abovementioned amount is used.

The process according to the invention is carried out in the absence of organosilicon compounds. “In the absence of organosilicon compounds, preferably in the absence of haloalkylsilanes” means that, for the entire duration of the reaction of the alkene, a proportion of not more than 0.1% by weight, preferably of not more than 0.02% by weight, of organosilicon compounds, preferably of not more than 0.1% by weight, preferably of 0.02% by weight, of haloalkylsilanes, based on the mass of the elemental zinc, is present in the reaction mixture. In an alternative embodiment, the process according to the invention is carried out without addition of organosilicon compounds, preferably without addition of haloalkylsilanes. “Without addition of organosilicon compounds” means for the purposes of the invention that no organosilicon compounds, preferably no haloalkylsilanes, were at any point during or prior to the reaction added to the alkene, preferably to the compound of formula (I), to the bromochloromethane, to the cyclopropyl compound, preferably to the compound of formula (II), to the elemental zinc, to the elemental copper and/or the copper(I) compounds and/or the copper(II) compounds, to the solvent, to further starting materials added to the reaction mixture or to the reaction mixture itself. A proportion of more than 0.1% by weight, preferably of more than 0.02% by weight, of organosilicon compounds based on the mass of the elemental zinc is conceivable in the reaction mixture only if a corresponding amount of organosilicon compounds, preferably of haloalkylsilanes, is added to the reaction mixture or to one of the starting materials before or during the reaction. Organosilicon compounds do not occur as natural impurities in the starting materials—alkene, bromochloromethane, elemental zinc, elemental copper and/or copper(I) compounds and/or copper(II) compounds and (iii) solvents. Organosilicon compounds are preferably haloalkylsilanes. Haloalkylsilanes are for example chlorotrialkylsilanes. Preferred representatives of chlorotrialkylsilanes are chlorotrimethylsilane, chlorotriethylsilane, chlorotributylsilane, chlorotriisobutylsilane or chlorotrihexylsilane.

Solvents that may be used in the process according to the invention are for example ethers and/or aromatic hydrocarbons. Preferred ethers are diethyl ether, 1,2-dimethoxyethane, methyl tert-butyl ether, tetrahydrofuran, cyclopentyl methyl ether, and mixtures thereof. A preferred aromatic hydrocarbon is toluene. From 0.7 to 1.5 mol, preferably from 1.0 to 1.2 mol, of ether and/or from 2.0 to 6.0 mol, preferably from 2.0 to 3.0 mol, of aromatic hydrocarbon per mol of alkene is preferably used.

The process according to the invention is described in more detail hereinbelow:

All steps in this reaction prior to hydrolysis of the reaction mixture are normally carried out under an inert gas atmosphere. Examples of suitable inert gases are nitrogen or argon.

In one embodiment of the process according to the invention, a reaction vessel is initially charged with the starting materials—alkene, preferably compound of formula (I), elemental zinc, elemental copper and/or copper(I) compounds and/or copper(II) compounds, and solvents—at temperatures of from 15 to 80° C., preferably from 50 to 70° C. This gives rise to a heterogeneous two-phase mixture that first undergoes mechanical or hydraulic mixing to obtain a two-phase mixture that is as homogeneous as possible. Since the mixing of the abovementioned starting materials is not exothermic at ambient temperature, the individual starting materials can be added discontinuously or continuously and in any order. Preferably, the solvent is initially charged and the other starting materials then added thereto with mixing. This can prevent the solid starting materials from clumping together. After or during the addition of the starting materials, the temperature can be increased to 85° C. Bromochloromethane is then added to the reaction mixture. This takes place for example discontinuously or continuously, preferably continuously. Preferably, 0.05 to 0.1 mol of bromochloromethane per mol of alkene, preferably compound of formula (I), is initially added to the reaction mixture. The reaction then commences within 1 to 240 minutes through an exothermic evolution of heat, as a result of which the temperature of the reaction mixture can rise by 1.5 to 10° C. Once the exothermic evolution of heat has commenced, the temperature of the reaction mixture is held within a range from 55 to 85° C., preferably within a range from 60 to 80° C., more preferably within a range from 67 to 73° C., by cooling, preferably by external cooling of the reaction vessel. The addition of the bromochloromethane is continued until the total amount of bromochloromethane has been added. During the addition of the bromochloromethane, it is possible to meter into the reaction mixture elemental copper and/or copper(I) compounds and/or copper(II) compounds if the exothermicity and thus the progress of the reaction declines. This normally brings the exothermicity, and thus the progress of the reaction, back up again. After the total amount of bromochloromethane has been added, the reaction mixture is further mixed for another 2 to 5 hours in a temperature range from 55 to 85° C., preferably in a range from 60 to 80° C., more preferably from 65 to 75° C. Preferably the reaction temperature is from 55 to 85° C., preferably from 60 to 80° C., and more preferably during and after the addition of bromochloromethane from 65 to 75° C. During this time, samples of the reaction mixture can be taken and, after working up, analyzed to determine the content of alkene, preferably of the alkene of formula (I), and/or of the cyclopropyl compound, preferably of the compound of formula (II). Once the reaction has ended, the reaction mixture, i.e. the mixture of all the starting materials added up to that point, is referred to as the crude mixture.

In a preferred embodiment of the process according to the invention, 0.01% to 5% by weight, preferably from 0.1% to 1% by weight, based on the total weight of the crude mixture, of a crude mixture from a previous reaction, based on the amount of alkene, preferably of the alkene of formula (I), used is added to the reaction mixture. It is normally sufficient to leave the remnants of the crude mixture from the previous reaction in the reactor. The crude mixture from a previous reaction corresponds to the reaction mixture after the reaction has ended, but before the mixture is hydrolyzed. The crude mixture from a previous reaction may either be added at the start of the addition of the starting materials—alkene, preferably compound of formula (I), elemental zinc, elemental copper and/or copper(I) compounds and/or copper(II) compounds, and solvents—to the reaction vessel, or is still present from a previous reaction in the same reaction vessel. However, it is also possible for the crude mixture from a previous reaction to be added at a later point in the mixing of the starting materials—alkene, preferably compound of formula (I), elemental zinc, elemental copper and/or copper(I) compounds and/or copper(II) compounds, and solvents. This promotes the commencement of the exothermic reaction. In a further embodiment, the commencement of the exothermic reaction can also be promoted by carrying out the process according to the invention reaction in the presence of 0.1 to 5% by weight of zinc halide, preferably zinc chloride or zinc bromide, based on the employed amount of the compound of formula (II). The zinc halide is preferably added at the start of the reaction.

The progress of the reaction can be determined by analyzing samples that have been worked up in the same way as the reaction mixture. The content of reactant and product can normally be determined by HPLC or gas chromatography, either as a percentage by area without external standard or as a percentage by weight with external standard.

Once the reaction has ended, the crude mixture is normally hydrolyzed. This is done for example by initially charging a separate reaction vessel with 1 to 5 kg of water and/or ice per kg of alkene used, preferably per kg of compound of formula (I) used, and adding to this the crude mixture, preferably with mechanical and/or hydraulic mixing. To this mixture is then added for example 0.5 to 1.5 mol of hydrogen chloride and/or hydrogen bromide, preferably in the form of 20 to 35% aqueous acid, per mole of alkene used, preferably per mole of compound of formula (I) used. The hydrolysis that occurs is exothermic. During the hydrolysis, it is preferable to ensure that the temperature of the mixture does not rise above 35° C. Once the hydrogen chloride has been added, the pH of the reaction mixture is for example from 6.5 to 7.5. If water is completely or partially replaced by ice, the chosen amount of ice is ideally such that no more ice is present in the reaction mixture at the end of the hydrolysis.

Once the hydrolysis has ended, the hydrolyzed reaction mixture is washed for example by adding a water-immiscible solvent, preferably an aromatic hydrocarbon, further preferably toluene, preferably with mechanical and/or hydraulic mixing. After phase separation into an organic phase, which contains the cyclopropanated product, and an aqueous phase, extraction of the aqueous phase can be repeated. The isolated organic phases are then preferably combined.

The organic phase containing the cyclopropyl compound, preferably the compound of formula (II), can either be used as such in a new reaction or else worked up further in order to isolate the cyclopropyl compound, preferably the compound of formula (II), therefrom.

This isolation takes place for example by distilling off solvent so that the cyclopropyl compound, preferably the compound of formula (II), is left behind as the bottoms. The process according to the invention surprisingly affords—even in the absence of organosilicon compounds, preferably in the absence of haloalkylsilanes, and/or without addition of organosilicon compounds, preferably without addition of haloalkylsilanes—the cyclopropyl compounds, preferably the compound of formula (II), in yields of 85 to 95% of theory.

### EXAMPLES

Example 1 (According to the Invention) [1-(4-chlorophenyl)-2-cyclopropylpropan-1-ol; Compound of Formula (II)]

All steps in this reaction prior to hydrolysis of the reaction mixture were carried out under a nitrogen atmosphere. A reactor was initially charged with 79.4 g (0.85 mol) of dimethoxyethane, 152.0 g of 4-(4-chlorophenyl)-3-methylbut-1-en-4-ol [compound of formula (I)] (content 97.0% by weight, 0.75 mol), 193.3 g (2.07 mol) of toluene, 120 g of zinc powder (1.84 mol), and 0.188 g (1.9 mmol) of copper(I) chloride at ambient temperature and also 1-2 g of unhydrolyzed reaction mixture from a previous reaction. The mixture was heated to 85° C. while stirring. On reaching this temperature, 7.25 g of bromochloromethane was metered in over the course of 10 minutes. After 6 minutes, an exothermic reaction commenced and the reaction mixture warmed by 2° C. The reaction mixture was then adjusted to a temperature range of 67 to 73° C. by cooling. 179.1 g (1.38 mol) of bromochloromethane was then metered into the reaction mixture such that the temperature of the reaction mixture continued to remain within a range from 67 to 73° C. This was accompanied by an escape of gaseous methyl chloride from the reaction mixture, which was discharged from the reactor into a scrubber via a stream of nitrogen. At the end of the metered addition, the reaction mixture was mixed for 3 hours by stirring. The temperature of the reaction mixture was then lowered to 45° C. with stirring. To hydrolyze the reaction mixture, the reaction mixture thus cooled was added with stirring to a mixture of 110 g of hydrochloric acid (30% by weight) and 500 g of water such that the pH of the aqueous phase of the two-phase mixture was 6.5 to 7.5. After phase separation, the first upper organic phase was separated off. The first lower aqueous phase was mixed with 50 g of toluene, stirred, and left to stand to allow the phases to separate. The second upper organic phase was separated off and combined with the first upper organic phase. The solvent was removed from the combined organic phase by distillation at 90° C. and 20 hPa, affording the product (1-(4-chlorophenyl)-2-cyclopropylpropan-1-01) (168.4 g, content: 85.4% by weight) as the crude product in a yield of 91% of theory.

TABLE 1
The experiments according to examples 2 and 3 were carried out in analogous manner
to the procedure for example 1, but with the parameters listed in the table.

| | | | | | Methylene | |
|---|---|---|---|---|---|---|
| | | Molar | | | component/ | Yield |
| | Reaction | equivalents | | Mol % of | molar | (% of |
| Example | temperature | of zinc *⁾ | Catalyst | catalyst *⁾ | equivalents *⁾ | theory) |
| 1 | 67 to | 2.45 | CuCl | 0.25 | Bromochloro- | 91 |
| | 73° C. | | | | methane/1.91 | |
| 2 | 85° C. | 2.45 | CuCl | 0.25 | Dibromo- | 82 |
| | | | | | methane/1.91 | |
| 3 | 85° C. | 2.45 | CuCl | 0.25 | Dichloro- | 0 |
| | | | | | methane/1.91 | |

*⁾ based on alkene

1. A process for preparing cyclopropyl compounds that comprises the reaction of the corresponding alkene, which contains at least one carbon-carbon double bond, with bromochloromethane in the presence of (i) elemental zinc, (ii) catalytically active amounts of elemental copper and/or copper(I) compounds and/or copper(II) compounds, and (iii) at least one solvent, wherein the reaction of the alkene takes place without addition of organosilicon compounds, and that the elemental zinc has a lead content of not more than 0.005% by weight.

2. The process for preparing cyclopropyl compounds as claimed in claim 1, wherein the alkene is a compound of formula (I), 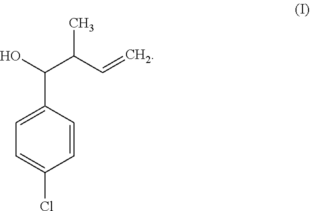
and the cyclopropyl compound obtained is a compound of formula (II),

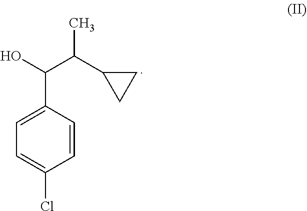

3. The process as claimed in claim 1, wherein the elemental zinc has a particle size distribution D90ₘₐₛₛ of not more than 0.5 mm.

4. The process as claimed in claim 1, wherein the elemental zinc has a total content of other metals of not more than 1% by weight.

5. The process as claimed in claim 1, wherein the elemental copper has a total content of other metals of not more than 1% by weight.

6. The process as claimed in claim 1, wherein the copper compounds are selected from copper(I) chloride, copper(II) chloride, copper(II) phosphate, copper(II) carbonate and mixtures thereof.

7. The process as claimed in claim 1, wherein the haloalkylsilane is a chlorotrialkylsilane.

8. The process as claimed in claim 1, wherein the chlorotrialkylsilane is selected from chlorotrimethylsilane, chlorotriethylsilane, chlorotributylsilane, chlorotriisobutylsilane, chlorotrihexylsilane, and mixtures thereof.

9. The process as claimed in claim 1, wherein the at least one solvent is an ether, selected from diethyl ether, 1,2-dimethoxyethane, methyl tert-butyl ether, tetrahydrofuran, cyclopentyl methyl ether, and mixtures thereof, and/or an aromatic hydrocarbon, toluene.

10. The process as claimed in claim 1, wherein the bromochloromethane has a purity of at least 98% by weight and/or a water content of less than 0.02% by weight.

11. The process as claimed in claim 1, wherein from 1.5 to 4 mol, of metallic zinc based on 1.0 mol of the alkene is used.

12. The process as claimed in claim 1, wherein the catalytically active amount of elemental copper and/or copper(I) compounds and/or copper(II) compounds is used in a total amount of from 0.001 to 0.1 mol, based on 1.0 mol of the alkene.

13. The process as claimed in claim 1, wherein from 1 to 3 mol of bromochloromethane based on 1.0 mol of the alkene is used.

14. The process as claimed in claim 1, wherein the reaction temperature is from 55 to 85° C., from 60 to 80° C.

15. The process as claimed in claim 1, wherein the alkene, compound of formula (I), bromochloromethane, elemental zinc, catalytically active amounts of elemental copper and/or copper(I) compounds and/or copper(II) compounds, and at least one solvent, are mixed together to prepare the cyclopropyl compound giving rise to a crude mixture, and the crude mixture is hydrolyzed at the end of the preparation.

16. The process as claimed in claim 1, wherein the preparation of the cyclopropyl compound takes place in the presence of 0.1% to 5% by weight, based on the total weight of the crude mixture, of a crude mixture from a previous preparation of the cyclopropyl compound, based on the amount of alkene used.
